(19) United States
(12) Patent Application Publication
(10) Pub. No.: US 2020/0347422 A1
(43) Pub. Date: Nov. 5, 2020

(54) PROCESS FOR ENZYMATIC HYDROLYSIS OF LIGNOCELLULOSIC MATERIAL AND FERMENTATION OF SUGARS

(71) Applicant: DSM IP ASSETS B.V., Heerlen (NL)

(72) Inventors: Maaike APPELDOORN, Echt (NL); Jozef Petrus Johannes SCHMITZ, Echt (NL); Bertus NOORDAM, Echt (NL)

(21) Appl. No.: 16/759,857

(22) PCT Filed: Oct. 29, 2018
(86) PCT No.: PCT/EP2018/079546

§ 371 Date: Apr. 28, 2020

(30) Foreign Application Priority Data
Oct. 30, 2017 (EP) 17199104.5

(51) Int. Cl. C12P 19/14; C12N 1/22; C12N 1/16

(52) U.S. Cl. CPC C12P 19/14; C12P 2201/00; C12N 1/16; C12N 1/22

(57) ABSTRACT

The invention relates to a process for the preparation of a sugar and/or fermentation product from lignocellulosic material.

### FIELD

The application relates to a process for preparing a sugar product from lignocellulosic material by enzymatic hydrolysis and a process for preparing a fermentation product by fermentation of sugars.

### BACKGROUND

Lignocellulosic material is primarily composed of cellulose, hemicellulose and lignin and provides an attractive platform for generating alternative energy sources to fossil fuels. The material is available in large amounts and can be converted into valuable products e.g. sugars or biofuel, such as bioethanol.

Producing fermentation products from lignocellulosic material is known in the art and generally includes the steps of pretreatment, hydrolysis, fermentation, and optionally recovery of the fermentation products.

During the hydrolysis, which may comprise the steps of liquefaction, pre-saccharification and/or saccharification, cellulose present in the lignocellulosic material is partly (typically 30 to 95%, dependable on enzyme activity and hydrolysis conditions) converted into sugars by cellulolytic enzymes. The hydrolysis typically takes place during a process lasting 6 to 168 hours (see Kumar, S., Chem. Eng. Technol. 32 (2009), 517-526) under elevated temperatures of 45 to 50° C. and non-sterile conditions.

Commonly, the sugars are then converted into valuable fermentation products such as ethanol by microorganisms like yeast. The fermentation takes place in a separate, preferably anaerobic, process step, either in the same or in a different vessel. The temperature during fermentation is adjusted to 30 to 33° C. to accommodate growth and ethanol production by microorganisms, commonly yeasts. During the fermentation process, the remaining cellulosic material is converted into sugars by the enzymes already present from the hydrolysis step, while microbial biomass and ethanol are produced. The fermentation is finished once the cellulosic material is converted into fermentable sugars and all fermentable sugars are converted into ethanol, carbon dioxide and microbial biomass. This may take up to 6 days. In general, the overall process time of hydrolysis and fermentation may amount up to 13 days.

In general, cost of enzyme production is a major cost factor in the overall production process of fermentation products from lignocellulosic material (see Kumar, S., Chem. Eng. Technol. 32 (2009), 517-526). Thus far, reduction of enzyme production costs is achieved by applying enzyme products from a single or from multiple microbial sources (see WO 2008/008793) with broader and/or higher (specific) hydrolytic activity. This leads to a lower enzyme need, faster conversion rates and/or higher conversion yields and thus to lower overall production costs.

Next to the optimization of enzymes, optimization of process design is a crucial tool to reduce overall costs of the production of sugar products and fermentation products.

For economic reasons, it is therefore desirable to include new and innovative process configurations aimed at reducing overall production costs in the process involving hydrolysis and fermentation of lignocellulosic material.

### SUMMARY

An object of the application is to provide an improved process for the preparation of a sugar product and/or a fermentation product from lignocellulosic material. The process is improved by treating the lignocellulosic material with an enzyme composition comprising a lytic polysaccharide monooxygenase. Thereafter, oxygen is added to the mixture comprising the lignocellulosic material and the enzyme composition and then additional lytic polysaccharide monooxygenase is added to the mixture comprising the lignocellulosic material and the enzyme composition.

### DETAILED DESCRIPTION

Throughout the present specification and the accompanying claims, the words “comprise” and “include” and variations such as “comprises”, “comprising”, “includes” and “including” are to be interpreted inclusively. That is, these words are intended to convey the possible inclusion of other elements or integers not specifically recited, where the context allows. The articles “a” and “an” are used herein to refer to one or to more than one (i.e. to one or at least one) of the grammatical object of the article. By way of example, “an element” may mean one element or more than one element.

The present application relates to a process for the preparation of a sugar product from lignocellulosic material, said process comprising the steps of (a) enzymatically hydrolysing lignocellulosic material to obtain the sugar product in a process comprising the steps of (i) treating the lignocellulosic material with an enzyme composition comprising a lytic polysaccharide monooxygenase, (ii) adding oxygen to the mixture comprising the lignocellulosic material and the enzyme composition, and (iii) adding additional lytic polysaccharide monooxygenase to the mixture comprising the lignocellulosic material and the enzyme composition, and (b) optionally, recovering the sugar product.

The present application also relates to a process for the preparation of a fermentation product from lignocellulosic material, comprising the steps of (a) performing a process for the preparation of a sugar product from lignocellulosic material as described herein, (b) fermenting the sugar product to produce the fermentation product, and (c) optionally, recovering the fermentation product.

In an embodiment the lignocellulosic material is pretreated before and/or during the enzymatic hydrolysis, preferably before enzymatic hydrolysis. Pretreatment methods are known in the art and include, but are not limited to, heat, mechanical, chemical modification, biological modification and any combination thereof. Pretreatment is typically performed in order to enhance the accessibility of the lignocellulosic material to enzymatic hydrolysis and/or hydrolyse the hemicellulose and/or solubilize the hemicellulose and/or cellulose and/or lignin, in the lignocellulosic material. In an embodiment, the pretreatment comprises treating the lignocellulosic material with steam explosion, hot water treatment or treatment with dilute acid or dilute base. Examples of pretreatment methods include, but are not limited to, steam treatment (e.g. treatment at 100-260° C., at a pressure of 7-45 bar, at neutral pH, for 1-10 minutes), dilute acid treatment (e.g. treatment with 0.1-5% H₂SO₄ and/or SO₂ and/or HNO₃ and/or HCl, in presence or absence of steam, at 120-200° C., at a pressure of 2-15 bar, at acidic pH, for 2-30 minutes), organosolv treatment (e.g. treatment with 1-1.5% H₂SO₄ in presence of organic solvent and steam, at 160-200° C., at a pressure of 7-30 bar, at acidic pH, for 30-60 minutes), lime treatment (e.g. treatment with 0.1-2% NaOH/Ca(OH)₂ in the presence of water/steam at 60-160° C., at a pressure of 1-10 bar, at alkaline pH, for 60-4800 minutes), ARP treatment (e.g. treatment with 5-15% NH₃, at 150-180° C., at a pressure of 9-17 bar, at alkaline pH, for 10-90 minutes), AFEX treatment (e.g. treatment with >15% NH₃, at 60-140° C., at a pressure of 8-20 bar, at alkaline pH, for 5-30 minutes).

The lignocellulosic material may be washed. In an embodiment the lignocellulosic material may be washed after the pretreatment. The washing step may be used to remove water soluble compounds that may act as inhibitors for the fermentation and/or hydrolysis step. The washing step may be conducted in manner known to the skilled person. Next to washing, other detoxification methods do exist. The lignocellulosic material may also be detoxified by any (or any combination) of these methods which include, but are not limited to, solid/liquid separation, vacuum evaporation, extraction, adsorption, neutralization, overliming, addition of reducing agents, addition of detoxifying enzymes such as laccases or peroxidases, addition of microorganisms capable of detoxification of hydrolysates. In an embodiment the enzymatically hydrolysed lignocellulosic material is washed and/or detoxified.

In the processes as described herein, lignocellulosic material may be added to a bioreactor and then enzymatically hydrolysed. In an embodiment the enzyme composition comprising a lytic polysaccharide monooxygenase is already present in the bioreactor before the lignocellulosic material is added. In another embodiment the enzyme composition comprising a lytic polysaccharide monooxygenase may be added to the bioreactor. In an embodiment the lignocellulosic material is already present in the bioreactor before the enzyme composition comprising a lytic polysaccharide monooxygenase is added. In an embodiment both the lignocellulosic material and the enzyme composition comprising a lytic polysaccharide monooxygenase are added simultaneously to the bioreactor. The enzyme composition comprising a lytic polysaccharide monooxygenase may be an aqueous composition.

In an embodiment the process for the preparation of a sugar product from lignocellulosic material comprises at least a liquefaction step wherein the lignocellulosic material is enzymatically hydrolysed in a first bioreactor, and at least a saccharification step wherein the liquefied lignocellulosic material is hydrolysed in the first bioreactor and/or in a second bioreactor. Saccharification can be done in the same bioreactor as the liquefaction (i.e. the first bioreactor). It can also be done in a separate bioreactor (i.e. the second bioreactor). In the enzymatic hydrolysis process liquefaction and saccharification may be separate steps. Alternatively, the liquefaction and saccharification may be combined. Liquefaction and saccharification may be performed at different temperatures, but may also be performed at a single temperature. In an embodiment the temperature of the liquefaction is higher than the temperature of the saccharification. Liquefaction is preferably carried out at a temperature of 60-75° C. and saccharification is preferably carried out at a temperature of 50-65° C. In an embodiment the enzyme composition comprising a lytic polysaccharide monooxygenase can be used in the liquefaction step and/or the saccharification step.

In an embodiment the enzymatic hydrolysis of the processes as described herein takes from 1 to 300 hours, from 2 to 250 hours, from 3 to 225 hours, from 4 to 200 hours, from 5 to 190 hours, from 10 to 180 hours, from 15 to 170 hours, from 20 to 160 hours and preferably from 25 to 150 hours.

In an embodiment oxygen is added during the process for the preparation of a sugar product from lignocellulosic material as described herein. In an embodiment the lignocellulosic material is first treated with an enzyme composition comprising a lytic polysaccharide monooxygenase and then oxygen is added to the mixture comprising the lignocellulosic material and the enzyme composition. In an embodiment the start of step (ii) of the process for the preparation of a sugar product from lignocellulosic material as described herein is from 1 to 100 hours after the start of step (i) of the process for the preparation of a sugar product from lignocellulosic material as described herein. This means that the lignocellulosic material is treated with an enzyme composition comprising a lytic polysaccharide monooxygenase and from 1 to 100 hours thereafter oxygen is added to the mixture comprising the lignocellulosic material and the enzyme composition. In an embodiment the start of step (ii) of the process for the preparation of a sugar product from lignocellulosic material as described herein is from 1 to 100 hours, from 5 to 95 hours, from 10 to 90 hours, from 15 to 85 hours, from 20 to 80 hours, preferably from 25 to 70 hours after the start of step (i) of the process for the preparation of a sugar product from lignocellulosic material as described herein.

Oxygen can be added continuously or discontinuously during the enzymatic hydrolysis. In an embodiment, when added discontinuously, oxygen can be added from 1%-10%, from 1%-15%, from 1%-20%, from 1%-25%, from 1%-30%, from 1%-35%, from 1%-40%, from 1%-45%, 1%-50%, from 1%-55%, from 1%-60%, from 1%-65%, from 1%-70%, from 1%-75%, from 1%-80%, from 1%-85%, from 1%-90%, from 1%-95%, or from 1%-99% of the total hydrolysis time. In an embodiment, when added in the second half of the hydrolysis process, oxygen can be added from 1%-10%, from 1%-15%, from 1%-20%, from 1%-25%, from 1%-30%, from 1%-35%, from 1%-40%, from 1%-45%, 1%-50%, from 1%-55%, from 1%-60%, from 1%-65%, from 1%-70%, from 1%-75%, from 1%-80%, from 1%-85%, from 1%-90%, from 1%-95%, or from 1%-99% of the time of the second half of the hydrolysis process. Oxygen can be added in several forms. For example, oxygen can be added as oxygen gas, oxygen-enriched gas, such as oxygen-enriched air, or air. Examples how to add oxygen include, but are not limited to, addition of oxygen by means of sparging, chemical addition of oxygen, filling the bioreactors used in the enzymatic hydrolysis from the top (plunging the hydrolysate into the bioreactor and consequently introducing oxygen into the hydrolysate) and addition of oxygen to the headspace of the bioreactors. In general, the amount of oxygen added to the bioreactors can be controlled and/or varied. Restriction of the oxygen supplied is possible by adding only oxygen during part of the hydrolysis time. Another option is adding oxygen at a low concentration, for example by using a mixture of air and recycled air (air leaving the bioreactor) or by “diluting” air with an inert gas. Increasing the amount of oxygen added can be achieved by addition of oxygen during longer periods of the hydrolysis time, by adding the oxygen at a higher concentration or by adding more air. Another way to control the oxygen concentration is to add an oxygen consumer and/or an oxygen generator. Oxygen can be introduced, for example blown, into the bioreactor, for example into the lignocellulosic material present in the bioreactor.

In an embodiment oxygen is added to the one or more bioreactors used in the enzymatic hydrolysis before and/or during and/or after the addition of the lignocellulosic material to the bioreactors. The oxygen may be introduced together with the lignocellulosic material that enters the bioreactor(s). The oxygen may be introduced into the material stream that will enter the bioreactor(s) or with part of the bioreactor(s) contents that passes an external loop of the bioreactor(s). Preferably, oxygen is added when the lignocellulosic material is in the bioreactor. Preferably, oxygen is added when the enzyme composition comprising a lytic polysaccharide monooxygenase is in the bioreactor. Preferably, oxygen is added when the lignocellulosic material and the enzyme composition comprising a lytic polysaccharide monooxygenase are in the bioreactor. Preferably, oxygen is added to the mixture comprising the lignocellulosic material and the enzyme composition. Preferably, the mixture is present in the bioreactor when the oxygen is added to it.

In an embodiment oxygen is added to the mixture comprising the lignocellulosic material and the enzyme composition such that the the level of dissolved oxygen (DO) in the mixture is maintained at a level of 0.1%-100% of the saturation dissolved oxygen level during the hydrolysis process. In an embodiment oxygen is added to the mixture comprising the lignocellulosic material and the enzyme composition such that the the level of dissolved oxygen in the mixture is maintained at a level of 2.5%-99% of the saturation dissolved oxygen level during the hydrolysis process. In an embodiment oxygen is added to the mixture comprising the lignocellulosic material and the enzyme composition such that the the level of dissolved oxygen in the mixture is maintained at a level of 5%-95% of the saturation dissolved oxygen level during the hydrolysis process. In an embodiment oxygen is added to the mixture comprising the lignocellulosic material and the enzyme composition such that the the level of dissolved oxygen in the mixture is maintained at a level of 7.5%-90% of the saturation dissolved oxygen level during the hydrolysis process. In an embodiment oxygen is added to the mixture comprising the lignocellulosic material and the enzyme composition such that the the level of dissolved oxygen in the mixture is maintained at a level of 10%-85% of the saturation dissolved oxygen level during the hydrolysis process. In an embodiment oxygen is added to the mixture comprising the lignocellulosic material and the enzyme composition such that the the level of dissolved oxygen in the mixture is maintained at a level of 13%-80% of the saturation dissolved oxygen level during the hydrolysis process. The DO can be measured using a DO probe. The probe can be immersed in the mixture held at the hydrolysis temperature. In an embodiment the probe has been precalibrated at the same temperature. The DO level can be monitored continuously or at intervals.

In an embodiment additional lytic polysaccharide monooxygenase is added during the process for the preparation of a sugar product from lignocellulosic material as described herein. In an embodiment the lignocellulosic material is first treated with an enzyme composition comprising a lytic polysaccharide monooxygenase, then oxygen is added to the mixture comprising the lignocellulosic material and the enzyme composition and thereafter additional lytic polysaccharide monooxygenase is added to the mixture comprising the lignocellulosic material and the enzyme composition comprising a lytic polysaccharide monooxygenase. During and/or after additional lytic polysaccharide monooxygenase is added to the mixture comprising the lignocellulosic material and the enzyme composition comprising a lytic polysaccharide monooxygenase, oxygen may still be added to the mixture. Alternatively, oxygen addition may be stopped during and/or after additional lytic polysaccharide monooxygenase is added to the mixture comprising the lignocellulosic material and the enzyme composition comprising a lytic polysaccharide monooxygenase.

In an embodiment additional lytic polysaccharide monooxygenase is added to the mixture comprising the lignocellulosic material and the enzyme composition (comprising a lytic polysaccharide monooxygenase) from 1 to 100 hours after the start of step (ii) of the process for the preparation of a sugar product from lignocellulosic material as described herein. In other words, step (iii) of the process for the preparation of a sugar product from lignocellulosic material as described herein starts from 1 to 100 hours after the start of step (ii) of the process for the preparation of a sugar product from lignocellulosic material as described herein.

In an embodiment the start of step (iii) of the process for the preparation of a sugar product from lignocellulosic material as described herein is from 1 to 100 hours, from 5 to 95 hours, from 10 to 90 hours, from 15 to 85 hours, from 20 to 80 hours, preferably from 25 to 70 hours after the start of step (ii) of the process for the preparation of a sugar product from lignocellulosic material as described herein.

In an embodiment the enzymatic hydrolysis is done in one or more bioreactors. In an embodiment the bioreactor(s) used in the processes as described herein have a volume of at least 1 m³. Preferably, the bioreactors have a volume of at least 2 m³, at least 3 m³, at least 4 m³, at least 5 m³, at least 6 m³, at least 7 m³, at least 8 m³, at least 9 m³, at least 10 m³, at least 15 m³, at least 20 m³, at least 25 m³, at least 30 m³, at least 35 m³, at least 40 m³, at least 45 m³, at least 50 m³, at least 60 m³, at least 70 m³, at least 75 m³, at least 80 m³, at least 90 m³, at least 100 m³, at least 200 m³, at least 300 m³, at least 400 m³, at least 500 m³, at least 600 m³, at least 700 m³, at least 800 m³, at least 900 m³, at least 1000 m³, at least 1500 m³, at least 2000 m³, at least 2500 m³. In general, the bioreactor(s) will be smaller than 3000 m³ or 5000 m³. In an embodiment the size of the bioreactor(s) is from 10 m³ to 5000 m³. In case multiple bioreactors are used in the enzymatic hydrolysis of the processes as described herein, they may have the same volume, but also may have a different volume.

In an embodiment the enzyme composition comprising a lytic polysaccharide monooxygenase and/or the additional lytic polysaccharide monooxygenase used in the processes as described herein is from a fungus, preferably a filamentous fungus. In an embodiment the enzymes in the enzyme composition as described herein are derived from a fungus, preferably a filamentous fungus or the enzymes comprise a fungal enzyme, preferably a filamentous fungal enzyme. The enzymes used in the enzymatic hydrolysis of the processes as described herein are derived from a fungus or the enzymes used in the enzymatic hydrolysis of the processes as described herein comprise a fungal enzyme. In an embodiment the lytic polysaccharide monooxygenase in the enzyme composition and/or the additional lytic polysaccharide monooxygenase are fungal lytic polysaccharide monooxygenases. In an embodiment the lytic polysaccharide monooxygenase in the enzyme composition and/or the additional lytic polysaccharide monooxygenase are identical. In another embodiment they differ.

“Filamentous fungi” include all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). Filamentous fungi include, but are not limited to Acremonium, Agaricus, Aspergillus, Aureobasidium, Beauvaria, Cephalosporium, Ceriporiopsis, Chaetomium paecilomyces, Chrysosporium, Claviceps, Cochiobolus, Coprinus, Cryptococcus, Cyathus, Emericella, Endothia, Endothia mucor, Filibasidium, Fusarium, Geosmithia, Gilocladium, Humicola, Magnaporthe, Mucor, Myceliophthora, Myrothecium, Neocallimastix, Neurospora, Paecilomyces, Penicillium, Piromyces, Panerochaete, Pleurotus, Podospora, Pyricularia, Rasamsonia, Rhizomucor, Rhizopus, Scylatidium, Schizophyllum, Stagonospora, Talaromyces, Thermoascus, Thermomyces, Thielavia, Tolypocladium, Trametes pleurotus, Trichoderma and Trichophyton. In a preferred embodiment the fungus is Rasamsonia, with Rasamsonia emersonii being most preferred. Ergo, the processes as described herein are advantageously applied in combination with enzymes derived from a microorganism of the genus Rasamsonia or the enzymes used in the processes as described herein comprise a Rasamsonia enzyme.

Several strains of filamentous fungi are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

The enzymatic hydrolysis processes as described herein are preferably done at 40-90° C. Preferably, the processes as described herein are done with thermostable enzymes. “Thermostable” enzyme as used herein means that the enzyme has a temperature optimum of 50° C. or higher, 60° C. or higher, 70° C. or higher, 75° C. or higher, 80° C. or higher, or even 85° C. or higher. They may for example be isolated from thermophilic microorganisms or may be designed by the skilled person and artificially synthesized. In one embodiment the polynucleotides encoding the thermostable enzymes may be isolated or obtained from thermophilic or thermotolerant filamentous fungi or isolated from non-thermophilic or non-thermotolerant fungi, but are found to be thermostable. By “thermophilic fungus” is meant a fungus that grows at a temperature of 50° C. or higher. By “themotolerant” fungus is meant a fungus that grows at a temperature of 45° C. or higher, having a maximum near 50° C.

Suitable thermophilic or thermotolerant fungal cells may be Humicola, Rhizomucor, Myceliophthora, Rasamsonia, Talaromyces, Thermomyces, Thermoascus or Thielavia cells, preferably Rasamsonia cells. Preferred thermophilic or thermotolerant fungi are Humicola grisea var. thermoidea, Humicola lanuginosa, Myceliophthora thermophila, Papulaspora thermophilia, Rasamsonia byssochlamydoides, Rasamsonia emersonii, Rasamsonia argillacea, Rasamsonia eburnean, Rasamsonia brevistipitata, Rasamsonia cylindrospora, Rhizomucor pusillus, Rhizomucor miehei, Talaromyces bacillisporus, Talaromyces leycettanus, Talaromyces thermophilus, Thermomyces lenuginosus, Thermoascus crustaceus, Thermoascus thermophilus Thermoascus aurantiacus and Thielavia terrestris.

Rasamsonia is a new genus comprising thermotolerant and thermophilic Talaromyces and Geosmithia species. Based on phenotypic, physiological and molecular data, the species Talaromyces emersonii, Talaromyces byssochlamydoides, Talaromyces eburneus, Geosmithia argillacea and Geosmithia cylindrospora were transferred to Rasamsonia gen. nov. Talaromyces emersonii, Penicillium geosmithia emersonii and Rasamsonia emersonii are used interchangeably herein.

In the processes as described herein enzyme compositions are used. Preferably, the compositions are stable. “Stable enzyme compositions” as used herein means that the enzyme compositions retain activity after 30 hours of hydrolysis reaction time, preferably at least 10%, 20%, 30%, 40%, 50%, 60%, 65%, 70%, 75%, 80% 85%, 90%, 95%, 96%, 97%, 98%, 99% or 100% of its initial activity after 30 hours of hydrolysis reaction time. Preferably, the enzyme composition retains activity after 40, 50, 60, 70, 80, 90 100, 150, 200, 250, 300, 350, 400, 450, 500 hours of hydrolysis reaction time.

The enzymes may be prepared by fermentation of a suitable substrate with a suitable microorganism, e.g. Rasamsonia emersonii or Aspergillus niger, wherein the enzymes are produced by the microorganism. The microorganism may be altered to improve or to make the enzymes. For example, the microorganism may be mutated by classical strain improvement procedures or by recombinant DNA techniques. Therefore, the microorganisms mentioned herein can be used as such to produce the enzymes or may be altered to increase the production or to produce altered enzymes which might include heterologous enzymes, e.g. cellulases, thus enzymes that are not originally produced by that microorganism. Preferably, a fungus, more preferably a filamentous fungus is used to produce the enzymes. Advantageously, a thermophilic or thermotolerant microorganism is used. Optionally, a substrate is used that induces the expression of the enzymes by the enzyme producing microorganism.

The enzymes are used to liquefy the lignocellulosic material and/or release sugars from lignocellulosic material that comprises polysaccharides. The major polysaccharides are cellulose (glucans), hemicelluloses (xylans, heteroxylans and xyloglucans). In addition, some hemicellulose may be present as glucomannans, for example in wood-derived lignocellulosic material. The enzymatic hydrolysis of these polysaccharides to soluble sugars, including both monomers and multimers, for example glucose, cellobiose, xylose, arabinose, galactose, fructose, mannose, rhamnose, ribose, galacturonic acid, glucoronic acid and other hexoses and pentoses occurs under the action of different enzymes acting in concert. By sugar product is meant the enzymatic hydrolysis product of the lignocellulosic material. The sugar product comprises soluble sugars, including both monomers and multimers. Preferably, it comprises glucose. Examples of other sugars are cellobiose, xylose, arabinose, galactose, fructose, mannose, rhamnose, ribose, galacturonic acid, glucoronic acid and other hexoses and pentoses. The sugar product may be used as such or may be further processed for example recovered and/or purified.

In addition, pectins and other pectic substances such as arabinans may make up considerably proportion of the dry mass of typically cell walls from non-woody plant tissues (about a quarter to half of dry mass may be pectins). Furthermore, the lignocellulosic material may comprise lignin.

In an embodiment the enzyme composition comprising a lytic polysaccharide monooxygenase and/or the additional lytic polysaccharide monooxygenase is added in the form of a whole fermentation broth of a fungus, preferably Rasamsonia. The whole fermentation broth can be prepared from fermentation of non-recombinant and/or recombinant filamentous fungi. In an embodiment the filamentous fungus is a recombinant filamentous fungus comprising one or more genes which can be homologous or heterologous to the filamentous fungus. In an embodiment, the filamentous fungus is a recombinant filamentous fungus comprising one or more genes which can be homologous or heterologous to the filamentous fungus wherein the one or more genes encode enzymes that can degrade a cellulosic substrate. The whole fermentation broth may comprise any of the enzymes described below or any combination thereof.

Preferably, the enzyme composition is a whole fermentation broth wherein the cells are killed. The whole fermentation broth may contain organic acid(s) (used for killing the cells), killed cells and/or cell debris, and culture medium.

Generally, the filamentous fungi are cultivated in a cell culture medium suitable for production of enzymes capable of hydrolyzing a cellulosic substrate. The cultivation takes place in a suitable nutrient medium comprising carbon and nitrogen sources and inorganic salts, using procedures known in the art. Suitable culture media, temperature ranges and other conditions suitable for growth and cellulase and/or hemicellulase and/or pectinase production are known in the art. The whole fermentation broth can be prepared by growing the filamentous fungi to stationary phase and maintaining the filamentous fungi under limiting carbon conditions for a period of time sufficient to express the one or more cellulases and/or hemicellulases and/or pectinases. Once enzymes, such as cellulases and/or hemicellulases and/or pectinases, are secreted by the filamentous fungi into the fermentation medium, the whole fermentation broth can be used. The whole fermentation broth of the present invention may comprise filamentous fungi. In some embodiments, the whole fermentation broth comprises the unfractionated contents of the fermentation materials derived at the end of the fermentation. Typically, the whole fermentation broth comprises the spent culture medium and cell debris present after the filamentous fungi is grown to saturation, incubated under carbon-limiting conditions to allow protein synthesis (particularly, expression of cellulases and/or hemicellulases and/or pectinases). In some embodiments, the whole fermentation broth comprises the spent cell culture medium, extracellular enzymes and filamentous fungi. In some embodiments, the filamentous fungi present in whole fermentation broth can be lysed, permeabilized, or killed using methods known in the art to produce a cell-killed whole fermentation broth. In an embodiment, the whole fermentation broth is a cell-killed whole fermentation broth, wherein the whole fermentation broth containing the filamentous fungi cells are lysed or killed. In some embodiments, the cells are killed by lysing the filamentous fungi by chemical and/or pH treatment to generate the cell-killed whole broth of a fermentation of the filamentous fungi. In some embodiments, the cells are killed by lysing the filamentous fungi by chemical and/or pH treatment and adjusting the pH of the cell-killed fermentation mix to a suitable pH. In an embodiment, the whole fermentation broth comprises a first organic acid component comprising at least one 1-5 carbon organic acid and/or a salt thereof and a second organic acid component comprising at least 6 or more carbon organic acid and/or a salt thereof. In an embodiment, the first organic acid component is acetic acid, formic acid, propionic acid, a salt thereof, or any combination thereof and the second organic acid component is benzoic acid, cyclohexanecarboxylic acid, 4-methylvaleric acid, phenylacetic acid, a salt thereof, or any combination thereof.

The term “whole fermentation broth” as used herein refers to a preparation produced by cellular fermentation that undergoes no or minimal recovery and/or purification. For example, whole fermentation broths are produced when microbial cultures are grown to saturation, incubated under carbon-limiting conditions to allow protein synthesis (e.g., expression of enzymes by host cells) and secretion into cell culture medium. Typically, the whole fermentation broth is unfractionated and comprises spent cell culture medium, extracellular enzymes, and microbial, preferably non-viable, cells.

If needed, the whole fermentation broth can be fractionated and the one or more of the fractionated contents can be used. For instance, the killed cells and/or cell debris can be removed from a whole fermentation broth to provide a composition that is free of these components.

The whole fermentation broth may further comprise a preservative and/or anti-microbial agent. Such preservatives and/or agents are known in the art.

The whole fermentation broth as described herein is typically a liquid, but may contain insoluble components, such as killed cells, cell debris, culture media components, and/or insoluble enzyme(s). In some embodiments, insoluble components may be removed to provide a clarified whole fermentation broth.

In an embodiment, the whole fermentation broth may be supplemented with one or more enzyme activities that are not expressed endogenously, or expressed at relatively low level by the filamentous fungi, to improve the degradation of the cellulosic substrate, for example, to fermentable sugars such as glucose or xylose. The supplemental enzyme(s) can be added as a supplement to the whole fermentation broth and the enzymes may be a component of a separate whole fermentation broth, or may be purified, or minimally recovered and/or purified.

In an embodiment, the whole fermentation broth comprises a whole fermentation broth of a fermentation of a recombinant filamentous fungus overexpressing one or more enzymes to improve the degradation of the cellulosic substrate. Alternatively, the whole fermentation broth can comprise a mixture of a whole fermentation broth of a fermentation of a non-recombinant filamentous fungus and a recombinant filamentous fungus overexpressing one or more enzymes to improve the degradation of the cellulosic substrate. In an embodiment, the whole fermentation broth comprises a whole fermentation broth of a fermentation of a filamentous fungus overexpressing beta-glucosidase. Alternatively, the whole fermentation broth for use in the present methods and reactive compositions can comprise a mixture of a whole fermentation broth of a fermentation of a non-recombinant filamentous fungus and a whole fermentation broth of a fermentation of a recombinant filamentous fungus overexpressing a beta-glucosidase.

In an embodiment the enzyme composition comprising a lytic polysaccharide monooxygenase further comprises a polypeptide selected from the group consisting of a cellobiohydrolase, an endoglucanase, a beta-glucosidase, a beta-xylosidase, an endoxylanase and any combination thereof. In an embodiment the additional lytic polysaccharide monooxygenase is added in the form of an enzyme composition. This enzyme composition may further comprise a polypeptide selected from the group consisting of a cellobiohydrolase, an endoglucanase, a beta-glucosidase, a beta-xylosidase, an endoxylanase and any combination thereof. The enzymes (that may be present in the enzyme compositions used in the processes as described herein) are described in more detail below. In another embodiment the additional lytic polysaccharide monooxygenase is added as a single enzyme. The single enzyme may be purified.

An enzyme composition for use in the processes as described herein may comprise at least two activities, although typically a composition will comprise more than two activities, for example, three, four, five, six, seven, eight, nine or even more activities. Typically, an enzyme composition for use in the processes as described herein comprises at least two cellulases. The at least two cellulases may contain the same or different activities. The enzyme composition for use in the processes as described herein may also comprises at least one enzyme other than a cellulase. Preferably, the at least one other enzyme has an auxiliary enzyme activity, i.e. an additional activity which, either directly or indirectly leads to lignocellulose degradation. Examples of such auxiliary activities are mentioned herein and include, but are not limited, to hemicellulases.

In an embodiment an enzyme composition for use in the hydrolysis processes as described herein comprises a lytic polysaccharide monooxygenase. In an embodiment the lytic polysaccharide monooxygenase added in step (i) of the process for the preparation of a sugar product from lignocellulosic material as described herein is identical to the additional lytic polysaccharide monooxygenase added in step (iii) of the process for the preparation of a sugar product from lignocellulosic material as described herein. In an embodiment the lytic polysaccharide monooxygenase added in step (i) of the process for the preparation of a sugar product from lignocellulosic material as described herein differs from the additional lytic polysaccharide monooxygenase added in step (iii) of the process for the preparation of a sugar product from lignocellulosic material as described herein. In an embodiment the lytic polysaccharide monooxygenase added in step (i) of the process for the preparation of a sugar product from lignocellulosic material as described herein and the additional lytic polysaccharide monooxygenase added in step (iii) of the process for the preparation of a sugar product from lignocellulosic material as described herein are both added in the form of a whole fermentation broth of a fungus. The whole fermentation broths may be the identical, but, alternatively, may also differ. In an embodiment the lytic polysaccharide monooxygenase added in step (i) of the process for the preparation of a sugar product from lignocellulosic material as described herein is added in the form of a whole fermentation broth of a fungus, while the additional lytic polysaccharide monooxygenase added in step (iii) of the process for the preparation of a sugar product from lignocellulosic material as described herein is added as a purified enzyme.

In an embodiment the ratio of lytic polysaccharide monooxygenase added in step (i) to lytic polysaccharide monooxygenase added in step (iii) is from 10:1 to 1:10, from 5:1 to 1:8, from 2:1 to 1:6, preferably from 2:1 to 1:4.

In an embodiment the enzyme composition comprising a lytic polysaccharide monooxygenase may comprise more than one lytic polysaccharide monooxygenase, i.e. comprises two or more different lytic polysaccharide monooxygenases, e.g. lytic polysaccharide monooxygenases from different fungi. In an embodiment the additional lytic polysaccharide monooxygenase added in step (iii) of the process for the preparation of a sugar product from lignocellulosic material as described herein may comprise more than one lytic polysaccharide monooxygenase, i.e. comprises two or more different lytic polysaccharide monooxygenases, e.g. lytic polysaccharide monooxygenases from different fungi.

An enzyme composition for use in the processes as described herein may comprise a lytic polysaccharide monooxygenase, an endoglucanase, a cellobiohydrolase and/or a beta-glucosidase. An enzyme composition may comprise more than one enzyme activity per activity class. For example, a composition may comprise two endoglucanases, for example an endoglucanase having endo-1,3(1,4)-β glucanase activity and an endoglucanase having endo-β-1,4-glucanase activity.

A composition for use in the processes as described herein may be derived from a fungus, such as a filamentous fungus, such as Rasamsonia, such as Rasamsonia emersonii. In an embodiment a core set of enzymes may be derived from Rasamsonia emersonii. If needed, the set of enzymes can be supplemented with additional enzymes from other sources. Such additional enzymes may be derived from classical sources and/or produced by genetically modified organisms.

In addition, enzymes in the enzyme compositions for use in the processes as described herein may be able to work at low pH. For the purposes of this invention, low pH indicates a pH of 5.5 or lower, 5 or lower, 4.9 or lower, 4.8 or lower, 4.7 or lower, 4.6 or lower, 4.5 or lower, 4.4 or lower, 4.3 or lower, 4.2 or lower, 4.1 or lower, 4.0 or lower 3.9 or lower, 3.8 or lower, 3.7 or lower, 3.6 or lower, 3.5 or lower.

An enzyme composition for use in the processes as described herein may comprise a cellulase and/or a hemicellulase and/or a pectinase from Rasamsonia. They may also comprise a cellulase and/or a hemicellulase and/or a pectinase from a source other than Rasamsonia. They may be used together with one or more Rasamsonia enzymes or they may be used without additional Rasamsonia enzymes being present.

An enzyme composition for use in the processes as described herein may comprise a lytic polysaccharide monooxygenas, an endoglucanase, one or two cellobiohydrolases and/or a beta-glucosidase.

An enzyme composition for use in the processes as described herein may comprise one type of cellulase activity and/or hemicellulase activity and/or pectinase activity provided by a composition as described herein and a second type of cellulase activity and/or hemicellulase activity and/or pectinase activity provided by an additional cellulase/hemicellulase/pectinase.

As used herein, a cellulase is any polypeptide which is capable of degrading or modifying cellulose. A polypeptide which is capable of degrading cellulose is one which is capable of catalyzing the process of breaking down cellulose into smaller units, either partially, for example into cellodextrins, or completely into glucose monomers. A cellulase according to the invention may give rise to a mixed population of cellodextrins and glucose monomers. Such degradation will typically take place by way of a hydrolysis reaction.

As used herein, a hemicellulase is any polypeptide which is capable of degrading or modifying hemicellulose. That is to say, a hemicellulase may be capable of degrading or modifying one or more of xylan, glucuronoxylan, arabinoxylan, glucomannan and xyloglucan. A polypeptide which is capable of degrading hemicellulose is one which is capable of catalyzing the process of breaking down the hemicellulose into smaller polysaccharides, either partially, for example into oligosaccharides, or completely into sugar monomers, for example hexose or pentose sugar monomers. A hemicellulase according to the invention may give rise to a mixed population of oligosaccharides and sugar monomers. Such degradation will typically take place by way of a hydrolysis reaction.

As used herein, a pectinase is any polypeptide which is capable of degrading or modifying pectin. A polypeptide which is capable of degrading pectin is one which is capable of catalyzing the process of breaking down pectin into smaller units, either partially, for example into oligosaccharides, or completely into sugar monomers. A pectinase according to the invention may give rise to a mixed population of oligosacchardies and sugar monomers. Such degradation will typically take place by way of a hydrolysis reaction.

Accordingly, an enzyme composition for use in the processes as described herein may comprise one or more of the following enzymes, a lytic polysaccharide monooxygenase (e.g. GH61), a cellobiohydrolase, an endoglucanase, and a beta-glucosidase. A composition for use in the processes as described herein may also comprise one or more hemicellulases, for example, an endoxylanase, a β-xylosidase, a α-L-arabionofuranosidase, an α-D-glucuronidase, an acetyl-xylan esterase, a feruloyl esterase, a coumaroyl esterase, an α-galactosidase, a β-galactosidase, a β-mannanase and/or a β-mannosidase. A composition for use in the processes as described herein may also comprise one or more pectinases, for example, an endo polygalacturonase, a pectin methyl esterase, an endo-galactanase, a beta galactosidase, a pectin acetyl esterase, an endo-pectin lyase, pectate lyase, alpha rhamnosidase, an exo-galacturonase, an expolygalacturonate lyase, a rhamnogalacturonan hydrolase, a rhamnogalacturonan lyase, a rhamnogalacturonan acetyl esterase, a rhamnogalacturonan galacturonohydrolase, and/or a xylogalacturonase. In addition, one or more of the following enzymes, an amylase, a protease, a lipase, a ligninase, a hexosyltransferase, a glucuronidase, an expansin, a cellulose induced protein or a cellulose integrating protein or like protein may be present in a composition for use in the processes as described herein (these are referred to as auxiliary activities above).

As used herein, lytic polysaccharide monooxygenases are enzymes that have recently been classified by CAZy in family AA9 (Auxiliary Activity Family 9) or family AA10 (Auxiliary Activity Family 10). Ergo, there exist AA9 lytic polysaccharide monooxygenases and AA10 lytic polysaccharide monooxygenases. Lytic polysaccharide monooxygenases are able to open a crystalline glucan structure and enhance the action of cellulases on lignocellulose substrates. They are enzymes having cellulolytic enhancing activity. Lytic polysaccharide monooxygenases may also affect cello-oligosaccharides. According to the latest literature, (see Isaksen et al., Journal of Biological Chemistry, vol. 289, no. 5, p. 2632-2642), proteins named GH61 (glycoside hydrolase family 61 or sometimes referred to EGIV) are lytic polysaccharide monooxygenases. GH61 was originally classified as endoglucanase based on measurement of very weak endo-1,4-β-d-glucanase activity in one family member, but have recently been reclassified by CAZy in family AA9. CBM33 (family 33 carbohydrate-binding module) is also a lytic polysaccharide monooxygenase (see Isaksen et al, Journal of Biological Chemistry, vol. 289, no. 5, pp. 2632-2642). CAZy has recently reclassified CBM33 in the AA10 family.

In an embodiment the lytic polysaccharide monooxygenase comprises an AA9 lytic polysaccharide monooxygenase. This means that at least one of the lytic polysaccharide monooxygenases in the enzyme composition and/or at least one of the additional lytic polysaccharide monooxygenases is an AA9 lytic polysaccharide monooxygenase. In an embodiment all lytic polysaccharide monooxygenases in the enzyme composition and/or all additional lytic polysaccharide monooxygenases are AA9 lytic polysaccharide monooxygenase.

In an embodiment the enzyme composition comprises a lytic polysaccharide monooxygenase from Thermoascus, such as Thermoascus aurantiacus, such as the one described in WO 2005/074656 as SEQ ID NO:2 and SEQ ID NO:1 in WO2014/130812 and in WO 2010/065830; or from Thielavia, such as Thielavia terrestris, such as the one described in WO 2005/074647 as SEQ ID NO: 8 or SEQ ID NO:4 in WO2014/130812 and in WO 2008/148131, and WO 2011/035027; or from Aspergillus, such as Aspergillus fumigatus, such as the one described in WO 2010/138754 as SEQ ID NO:2 or SEQ ID NO: 3 in WO2014/130812; or from Penicillium, such as Penicillium emersonii, such as the one disclosed as SEQ ID NO:2 in WO 2011/041397 or SEQ ID NO:2 in WO2014/130812. Other suitable lytic polysaccharide monooxygenases include, but are not limited to, Trichoderma reesei (see WO 2007/089290), Myceliophthora thermophila (see WO 2009/085935, WO 2009/085859, WO 2009/085864, WO 2009/085868), Penicillium pinophilum (see WO 2011/005867), Thermoascus sp. (see WO 2011/039319), and Thermoascus crustaceous (see WO 2011/041504). Other cellulolytic enzymes that may be comprised in the enzyme composition are described in WO 98/13465, WO 98/015619, WO 98/015633, WO 99/06574, WO 99/10481, WO 99/025847, WO 99/031255, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. Nos. 5,457,046, 5,648,263, and 5,686,593, to name just a few. In a preferred embodiment, the lytic polysaccharide monooxygenase is from Rasamsonia, e.g. Rasamsonia emersonii (see WO 2012/000892).

In an embodiment the additional lytic polysaccharide monooxygenase comprises one of the above-mentioned lytic polysaccharide monooxygenases.

As used herein, endoglucanases are enzymes which are capable of catalyzing the endohydrolysis of 1,4-β-D-glucosidic linkages in cellulose, lichenin or cereal β-D-glucans. They belong to EC 3.2.1.4 and may also be capable of hydrolyzing 1,4-linkages in β-D-glucans also containing 1,3-linkages. Endoglucanases may also be referred to as cellulases, avicelases, β-1,4-endoglucan hydrolases, β-1,4-glucanases, carboxymethyl cellulases, celludextrinases, endo-1,4-β-D-glucanases, endo-1,4-β-D-glucanohydrolases or endo-1,4-β-glucanases.

In an embodiment the endoglucanase comprises a GH5 endoglucanase and/or a GH7 endoglucanase. This means that at least one of the endoglucanases in the enzyme composition is a GH5 endoglucanase or a GH7 endoglucanase. In case there are more endoglucanases in the enzyme composition, these endoglucanases can be GH5 endoglucanases, GH7 endoglucanases or a combination of GH5 endoglucanases and GH7 endoglucanases. In a preferred embodiment the endoglucanase comprises a GH5 endoglucanase.

In an embodiment an enzyme composition as described herein comprises an endoglucanase from Trichoderma, such as Trichoderma reesei; from Humicola, such as a strain of Humicola insolens; from Aspergillus, such as Aspergillus aculeatus or Aspergillus kawachii; from Erwinia, such as Erwinia carotovara; from Fusarium, such as Fusarium oxysporum; from Thielavia, such as Thielavia terrestris; from Humicola, such as Humicola grisea var. thermoidea or Humicola insolens; from Melanocarpus, such as Melanocarpus albomyces; from Neurospora, such as Neurospora crassa; from Myceliophthora, such as Myceliophthora thermophila; from Cladorrhinum, such as Cladorrhinum foecundissimum; and/or from Chrysosporium, such as a strain of Chrysosporium lucknowense. In a preferred embodiment the endoglucanase is from Rasamsonia, such as a strain of Rasamsonia emersonii (see WO 01/70998). In an embodiment even a bacterial endoglucanase can be used including, but are not limited to, Acidothermus cellulolyticus endoglucanase (see WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (see WO 05/093050); and Thermobifida fusca endoglucanase V (see WO 05/093050).

As used herein, beta-xylosidases (EC 3.2.1.37) are polypeptides which are capable of catalysing the hydrolysis of 1,4-β-D-xylans, to remove successive D-xylose residues from the non-reducing termini. Beta-xylosidases may also hydrolyze xylobiose. Beta-xylosidase may also be referred to as xylan 1,4-β-xylosidase, 1,4-β-D-xylan xylohydrolase, exo-1,4-β-xylosidase or xylobiase.

In an embodiment the beta-xylosidase comprises a GH3 beta-xylosidase. This means that at least one of the beta-xylosidases in the enzyme composition is a GH3 beta-xylosidase. In an embodiment all beta-xylosidases in the enzyme composition are GH3 beta-xylosidases.

In an embodiment an enzyme composition as described herein comprises a beta-xylosidase from Neurospora crassa, Aspergillus fumigatus or Trichoderma reesei. In a preferred embodiment the enzyme composition comprises a beta-xylosidase from Rasamsonia, such as Rasamsonia emersonii (see WO 2014/118360).

As used herein, an endoxylanase (EC 3.2.1.8) is any polypeptide which is capable of catalysing the endohydrolysis of 1,4-β-D-xylosidic linkages in xylans. This enzyme may also be referred to as endo-1,4-β-xylanase or 1,4-β-D-xylan xylanohydrolase. An alternative is EC 3.2.1.136, a glucuronoarabinoxylan endoxylanase, an enzyme that is able to hydrolyze 1,4 xylosidic linkages in glucuronoarabinoxylans.

In an embodiment the endoxylanase comprises a GH10 xylanase. This means that at least one of the endoxylanases in the enzyme composition is a GH10 xylanase. In an embodiment all endoxylanases in the enzyme composition are GH10 xylanases.

In an embodiment an enzyme composition as described herein comprises an endoxylanase from Aspergillus aculeatus (see WO 94/21785), Aspergillus fumigatus (see WO 2006/078256), Penicillium pinophilum (see WO 2011/041405), Penicillium sp. (see WO 2010/126772), Thielavia terrestris NRRL 8126 (see WO 2009/079210), Talaromyces leycettanus, Thermobifida fusca, or Trichophaea saccata GH10 (see WO 2011/057083). In a preferred embodiment the enzyme composition comprises an endoxylanase from Rasamsonia, such as Rasamsonia emersonii (see WO 02/24926).

As used herein, a beta-glucosidase (EC 3.2.1.21) is any polypeptide which is capable of catalysing the hydrolysis of terminal, non-reducing β-D-glucose residues with release of β-D-glucose. Such a polypeptide may have a wide specificity for β-D-glucosides and may also hydrolyze one or more of the following: a β-D-galactoside, an α-L-arabinoside, a β-D-xyloside or a β-D-fucoside. This enzyme may also be referred to as amygdalase, β-D-glucoside glucohydrolase, cellobiase or gentobiase.

In an embodiment an enzyme composition as described herein comprises a beta-glucosidase from Aspergillus, such as Aspergillus oryzae, such as the one disclosed in WO 02/095014 or the fusion protein having beta-glucosidase activity disclosed in WO 2008/057637, or Aspergillus fumigatus, such as the one disclosed as SEQ ID NO:2 in WO 2005/047499 or SEQ ID NO:5 in WO 2014/130812 or an Aspergillus fumigatus beta-glucosidase variant, such as one disclosed in WO 2012/044915, such as one with the following substitutions: F100D, S283G, N456E, F512Y (using SEQ ID NO: 5 in WO 2014/130812 for numbering), or Aspergillus aculeatus, Aspergillus niger or Aspergillus kawachi. In another embodiment the beta-glucosidase is derived from Penicillium, such as Penicillium brasilianum disclosed as SEQ ID NO:2 in WO 2007/019442, or from Trichoderma, such as Trichoderma reesei, such as ones described in U.S. Pat. Nos. 6,022,725, 6,982,159, 7,045,332, 7,005,289, US 2006/0258554 US 2004/0102619. In an embodiment even a bacterial beta-glucosidase can be used. In another embodiment the beta-glucosidase is derived from Thielavia terrestris (WO 2011/035029) or Trichophaea saccata (WO 2007/019442). In a preferred embodiment the enzyme composition comprises a beta-glucosidase from Rasamsonia, such as Rasamsonia emersonii (see WO 2012/000886).

As used herein, a cellobiohydrolase (EC 3.2.1.91) is any polypeptide which is capable of catalyzing the hydrolysis of 1,4-β-D-glucosidic linkages in cellulose or cellotetraose, releasing cellobiose from the ends of the chains. This enzyme may also be referred to as cellulase 1,4-β-cellobiosidase, 1,4-β-cellobiohydrolase, 1,4-β-D-glucan cellobiohydrolase, avicelase, exo-1,4-β-D-glucanase, exocellobiohydrolase or exoglucanase.

In an embodiment an enzyme composition as described herein comprises a cellobiohydrolase I from Aspergillus, such as Aspergillus fumigatus, such as the Cel7A CBH I disclosed in SEQ ID NO:6 in WO 2011/057140 or SEQ ID NO:6 in WO 2014/130812; from Trichoderma, such as Trichoderma reesei; from Chaetomium, such as Chaetomium thermophilum; from Talaromyces, such as Talaromyces leycettanus or from Penicillium, such as Penicillium emersonii. In a preferred embodiment the enzyme composition comprises a cellobiohydrolase I from Rasamsonia, such as Rasamsonia emersonii (see WO 2010/122141).

In an embodiment an enzyme composition as described herein comprises a cellobiohydrolase II from Aspergillus, such as Aspergillus fumigatus, such as the one in SEQ ID NO:7 in WO 2014/130812 or from Trichoderma, such as Trichoderma reesei, or from Talaromyces, such as Talaromyces leycettanus, or from Thielavia, such as Thielavia terrestris, such as cellobiohydrolase II CEL6A from Thielavia terrestris. In a preferred embodiment the enzyme composition comprises a cellobiohydrolase II from Rasamsonia, such as Rasamsonia emersonii (see WO 2011/098580).

In an embodiment an enzyme composition as described herein comprises at least two cellulases. The at least two cellulases may contain the same or different activities. The enzyme composition may also comprise at least one enzyme other than a cellulase, e.g. a hemicellulase or a pectinase. In an embodiment the enzyme composition as described herein comprises one, two, three, four classes or more of cellulase, for example one, two, three or four or all of a lytic polysaccharide monooxygenase, an endoglucanase, one or two cellobiohydrolases and a beta-glucosidase.

In an embodiment an enzyme composition as described herein comprises a lytic polysaccharide monooxygenase, an endoglucanase, a cellobiohydrolase I, a cellobiohydrolase II, a beta-glucosidase, a beta-xylosidase and an endoxylanase.

In an embodiment an enzyme composition as described herein also comprises one or more of the below mentioned enzymes.

As used herein, a β-(1,3)(1,4)-glucanase (EC 3.2.1.73) is any polypeptide which is capable of catalysing the hydrolysis of 1,4-β-D-glucosidic linkages in β-D-glucans containing 1,3- and 1,4-bonds. Such a polypeptide may act on lichenin and cereal β-D-glucans, but not on β-D-glucans containing only 1,3- or 1,4-bonds. This enzyme may also be referred to as icheninase, 1,3-1,4-β-D-glucan 4-glucanohydrolase, β-glucanase, endo-β-1,3-1,4 glucanase, lichenase or mixed linkage β-glucanase. An alternative for this type of enzyme is EC 3.2.1.6, which is described as endo-1,3(4)-beta-glucanase. This type of enzyme hydrolyses 1,3- or 1,4-linkages in beta-D-glucanse when the glucose residue whose reducing group is involved in the linkage to be hydrolysed is itself substituted at C-3. Alternative names include endo-1,3-beta-glucanase, laminarinase, 1,3-(1,3;1,4)-beta-D-glucan 3 (4) glucanohydrolase. Substrates include laminarin, lichenin and cereal beta-D-glucans.

As used herein, an α-L-arabinofuranosidase (EC 3.2.1.55) is any polypeptide which is capable of acting on α-L-arabinofuranosides, α-L-arabinans containing (1,2) and/or (1,3)- and/or (1,5)-linkages, arabinoxylans and arabinogalactans. This enzyme may also be referred to as α-N-arabinofuranosidase, arabinofuranosidase or arabinosidase. Examples of arabinofuranosidases that may be comprised in the enzyme composition include, but are not limited to, arabinofuranosidases from Aspergillus niger, Humicola insolens DSM 1800 (see WO 2006/114094 and WO 2009/073383) and M. giganteus (see WO 2006/114094).

As used herein, an α-D-glucuronidase (EC 3.2.1.139) is any polypeptide which is capable of catalysing a reaction of the following form: alpha-D-glucuronoside+H(2)O=an alcohol+D-glucuronate. This enzyme may also be referred to as alpha-glucuronidase or alpha-glucosiduronase. These enzymes may also hydrolyse 4-O-methylated glucoronic acid, which can also be present as a substituent in xylans. An alternative is EC 3.2.1.131: xylan alpha-1,2-glucuronosidase, which catalyses the hydrolysis of alpha-1,2-(4-O-methyl)glucuronosyl links. Examples of alpha-glucuronidases that may be comprised in the enzyme composition include, but are not limited to, alpha-glucuronidases from Aspergillus clavatus, Aspergillus fumigatus, Aspergillus niger, Aspergillus terreus, Humicola insolens (see WO 2010/014706), Penicillium aurantiogriseum (see WO 2009/068565) and Trichoderma reesei.

As used herein, an acetyl-xylan esterase (EC 3.1.1.72) is any polypeptide which is capable of catalysing the deacetylation of xylans and xylo-oligosaccharides. Such a polypeptide may catalyze the hydrolysis of acetyl groups from polymeric xylan, acetylated xylose, acetylated glucose, alpha-napthyl acetate or p-nitrophenyl acetate but, typically, not from triacetylglycerol. Such a polypeptide typically does not act on acetylated mannan or pectin. Examples of acetylxylan esterases that may be comprised in the enzyme composition include, but are not limited to, acetylxylan esterases from Aspergillus aculeatus (see WO 2010/108918), Chaetomium globosum, Chaetomium gracile, Humicola insolens DSM 1800 (see WO 2009/073709), Hypocrea jecorina (see WO 2005/001036), Myceliophtera thermophila (see WO 2010/014880), Neurospora crassa, Phaeosphaeria nodorum and Thielavia terrestris NRRL 8126 (see WO 2009/042846). In a preferred embodiment the enzyme composition comprises an acetyl xylan esterase from Rasamsonia, such as Rasamsonia emersonii (see WO 2010/000888)

As used herein, a feruloyl esterase (EC 3.1.1.73) is any polypeptide which is capable of catalysing a reaction of the form: feruloyl-saccharide+H₂O=ferulate+saccharide. The saccharide may be, for example, an oligosaccharide or a polysaccharide. It may typically catalyse the hydrolysis of the 4-hydroxy-3-methoxycinnamoyl (feruloyl) group from an esterified sugar, which is usually arabinose in ‘natural’ substrates. p-nitrophenol acetate and methyl ferulate are typically poorer substrates. This enzyme may also be referred to as cinnamoyl ester hydrolase, ferulic acid esterase or hydroxycinnamoyl esterase. It may also be referred to as a hemicellulase accessory enzyme, since it may help xylanases and pectinases to break down plant cell wall hemicellulose and pectin. Examples of feruloyl esterases (ferulic acid esterases) that may be comprised in the enzyme composition include, but are not limited to, feruloyl esterases form Humicola insolens DSM 1800 (see WO 2009/076122), Neosartorya fischeri, Neurospora crassa, Penicillium aurantiogriseum (see WO 2009/127729), and Thielavia terrestris (see WO 2010/053838 and WO 2010/065448).

As used herein, a coumaroyl esterase (EC 3.1.1.73) is any polypeptide which is capable of catalysing a reaction of the form: coumaroyl-saccharide+H(2)O=coumarate+saccharide. The saccharide may be, for example, an oligosaccharide or a polysaccharide. This enzyme may also be referred to as trans-4-coumaroyl esterase, trans-p-coumaroyl esterase, p-coumaroyl esterase or p-coumaric acid esterase. This enzyme also falls within EC 3.1.1.73 so may also be referred to as a feruloyl esterase.

As used herein, an α-galactosidase (EC 3.2.1.22) is any polypeptide which is capable of catalysing the hydrolysis of terminal, non-reducing α-D-galactose residues in α-D-galactosides, including galactose oligosaccharides, galactomannans, galactans and arabinogalactans. Such a polypeptide may also be capable of hydrolyzing α-D-fucosides. This enzyme may also be referred to as melibiase.

As used herein, a β-galactosidase (EC 3.2.1.23) is any polypeptide which is capable of catalysing the hydrolysis of terminal non-reducing β-D-galactose residues in β-D-galactosides. Such a polypeptide may also be capable of hydrolyzing α-L-arabinosides. This enzyme may also be referred to as exo-(1->4)-β-D-galactanase or lactase.

As used herein, a β-mannanase (EC 3.2.1.78) is any polypeptide which is capable of catalysing the random hydrolysis of 1,4-β-D-mannosidic linkages in mannans, galactomannans and glucomannans. This enzyme may also be referred to as mannan endo-1,4-β-mannosidase or endo-1,4-mannanase.

As used herein, a β-mannosidase (EC 3.2.1.25) is any polypeptide which is capable of catalysing the hydrolysis of terminal, non-reducing β-D-mannose residues in β-D-mannosides. This enzyme may also be referred to as mannanase or mannase.

As used herein, an endo-polygalacturonase (EC 3.2.1.15) is any polypeptide which is capable of catalysing the random hydrolysis of 1,4-α-D-galactosiduronic linkages in pectate and other galacturonans. This enzyme may also be referred to as polygalacturonase pectin depolymerase, pectinase, endopolygalacturonase, pectolase, pectin hydrolase, pectin polygalacturonase, poly-α-1,4-galacturonide glycanohydrolase, endogalacturonase; endo-D-galacturonase or poly(1,4-α-D-galacturonide) glycanohydrolase.

As used herein, a pectin methyl esterase (EC 3.1.1.11) is any enzyme which is capable of catalysing the reaction: pectin+n H₂O=n methanol+pectate. The enzyme may also be known as pectinesterase, pectin demethoxylase, pectin methoxylase, pectin methylesterase, pectase, pectinoesterase or pectin pectylhydrolase.

As used herein, an endo-galactanase (EC 3.2.1.89) is any enzyme capable of catalysing the endohydrolysis of 1,4-β-D-galactosidic linkages in arabinogalactans. The enzyme may also be known as arabinogalactan endo-1,4-β-galactosidase, endo-1,4-β-galactanase, galactanase, arabinogalactanase or arabinogalactan 4-β-D-galactanohydrolase.

As used herein, a pectin acetyl esterase is defined herein as any enzyme which has an acetyl esterase activity which catalyses the deacetylation of the acetyl groups at the hydroxyl groups of GalUA residues of pectin.

As used herein, an endo-pectin lyase (EC 4.2.2.10) is any enzyme capable of catalysing the eliminative cleavage of (1→4)-α-D-galacturonan methyl ester to give oligosaccharides with 4-deoxy-6-O-methyl-α-D-galact-4-enuronosyl groups at their non-reducing ends. The enzyme may also be known as pectin lyase, pectin trans-eliminase; endo-pectin lyase, polymethylgalacturonic transeliminase, pectin methyltranseliminase, pectolyase, PL, PNL or PMGL or (1→4)-6-O-methyl-α-D-galacturonan lyase.

As used herein, a pectate lyase (EC 4.2.2.2) is any enzyme capable of catalysing the eliminative cleavage of (1→4)-α-D-galacturonan to give oligosaccharides with 4-deoxy-α-D-galact-4-enuronosyl groups at their non-reducing ends. The enzyme may also be known polygalacturonic transeliminase, pectic acid transeliminase, polygalacturonate lyase, endopectin methyltranseliminase, pectate transeliminase, endogalacturonate transeliminase, pectic acid lyase, pectic lyase, α-1,4-D-endopolygalacturonic acid lyase, PGA lyase, PPase-N, endo-α-1,4-polygalacturonic acid lyase, polygalacturonic acid lyase, pectin trans-eliminase, polygalacturonic acid trans-eliminase or (1→4)-α-D-galacturonan lyase.

As used herein, an alpha rhamnosidase (EC 3.2.1.40) is any polypeptide which is capable of catalysing the hydrolysis of terminal non-reducing α-L-rhamnose residues in α-L-rhamnosides or alternatively in rhamnogalacturonan. This enzyme may also be known as α-L-rhamnosidase T, α-L-rhamnosidase N or α-L-rhamnoside rhamnohydrolase.

As used herein, exo-galacturonase (EC 3.2.1.82) is any polypeptide capable of hydrolysis of pectic acid from the non-reducing end, releasing digalacturonate. The enzyme may also be known as exo-poly-α-galacturonosidase, exopolygalacturonosidase or exopolygalacturanosidase.

As used herein, exo-galacturonase (EC 3.2.1.67) is any polypeptide capable of catalysing: (1,4-α-D-galacturonide)ₙ+H₂O=(1,4-α-D-galacturonide)ₙ₋₁+D-galacturonate. The enzyme may also be known as galacturan 1,4-α-galacturonidase, exopolygalacturonase, poly(galacturonate) hydrolase, exo-D-galacturonase, exo-D-galacturonanase, exopoly-D-galacturonase or poly(1,4-α-D-galacturonide) galacturonohydrolase.

As used herein, exopolygalacturonate lyase (EC 4.2.2.9) is any polypeptide capable of catalysing eliminative cleavage of 4-(4-deoxy-α-D-galact-4-enuronosyl)-D-galacturonate from the reducing end of pectate, i.e. de-esterified pectin. This enzyme may be known as pectate disaccharide-lyase, pectate exo-lyase, exopectic acid transeliminase, exopectate lyase, exopolygalacturonic acid-trans-eliminase, PATE, exo-PATE, exo-PGL or (1→4)-α-D-galacturonan reducing-end-disaccharide-lyase.

As used herein, rhamnogalacturonan hydrolase is any polypeptide which is capable of hydrolyzing the linkage between galactosyluronic acid and rhamnopyranosyl in an endo-fashion in strictly alternating rhamnogalacturonan structures, consisting of the disaccharide [(1,2-alpha-L-rhamnoyl-(1,4)-alpha-galactosyluronic acid].

As used herein, rhamnogalacturonan lyase is any polypeptide which is any polypeptide which is capable of cleaving α-L-Rhap-(1→4)-α-D-GalpA linkages in an endo-fashion in rhamnogalacturonan by beta-elimination.

As used herein, rhamnogalacturonan acetyl esterase is any polypeptide which catalyzes the deacetylation of the backbone of alternating rhamnose and galacturonic acid residues in rhamnogalacturonan.

As used herein, rhamnogalacturonan galacturonohydrolase is any polypeptide which is capable of hydrolyzing galacturonic acid from the non-reducing end of strictly alternating rhamnogalacturonan structures in an exo-fashion.

As used herein, xylogalacturonase is any polypeptide which acts on xylogalacturonan by cleaving the 3-xylose substituted galacturonic acid backbone in an endo-manner. This enzyme may also be known as xylogalacturonan hydrolase.

As used herein, an α-L-arabinofuranosidase (EC 3.2.1.55) is any polypeptide which is capable of acting on α-L-arabinofuranosides, α-L-arabinans containing (1,2) and/or (1,3)- and/or (1,5)-linkages, arabinoxylans and arabinogalactans. This enzyme may also be referred to as α-N-arabinofuranosidase, arabinofuranosidase or arabinosidase.

As used herein, endo-arabinanase (EC 3.2.1.99) is any polypeptide which is capable of catalysing endohydrolysis of 1,5-α-arabinofuranosidic linkages in 1,5-arabinans. The enzyme may also be known as endo-arabinase, arabinan endo-1,5-α-L-arabinosidase, endo-1,5-α-L-arabinanase, endo-α-1,5-arabanase; endo-arabanase or 1,5-α-L-arabinan 1,5-α-L-arabinanohydrolase.

“Protease” includes enzymes that hydrolyze peptide bonds (peptidases), as well as enzymes that hydrolyze bonds between peptides and other moieties, such as sugars (glycopeptidases). Many proteases are characterized under EC 3.4 and are suitable for use in the processes as described herein. Some specific types of proteases include, cysteine proteases including pepsin, papain and serine proteases including chymotrypsins, carboxypeptidases and metalloendopeptidases.

“Lipase” includes enzymes that hydrolyze lipids, fatty acids, and acylglycerides, including phospoglycerides, lipoproteins, diacylglycerols, and the like. In plants, lipids are used as structural components to limit water loss and pathogen infection. These lipids include waxes derived from fatty acids, as well as cutin and suberin.

“Ligninase” includes enzymes that can hydrolyze or break down the structure of lignin polymers. Enzymes that can break down lignin include lignin peroxidases, manganese peroxidases, laccases and feruloyl esterases, and other enzymes described in the art known to depolymerize or otherwise break lignin polymers. Also included are enzymes capable of hydrolyzing bonds formed between hemicellulosic sugars (notably arabinose) and lignin. Ligninases include but are not limited to the following group of enzymes: lignin peroxidases (EC 1.11.1.14), manganese peroxidases (EC 1.11.1.13), laccases (EC 1.10.3.2) and feruloyl esterases (EC 3.1.1.73).

“Hexosyltransferase” (2.4.1-) includes enzymes which are capable of catalysing a transferase reaction, but which can also catalyze a hydrolysis reaction, for example of cellulose and/or cellulose degradation products. An example of a hexosyltransferase which may be used is a ß-glucanosyltransferase. Such an enzyme may be able to catalyze degradation of (1,3)(1,4)glucan and/or cellulose and/or a cellulose degradation product.

“Glucuronidase” includes enzymes that catalyze the hydrolysis of a glucuronoside, for example β-glucuronoside to yield an alcohol. Many glucuronidases have been characterized and may be suitable for use, for example β-glucuronidase (EC 3.2.1.31), hyalurono-glucuronidase (EC 3.2.1.36), glucuronosyl-disulfoglucosamine glucuronidase (3.2.1.56), glycyrrhizinate β-glucuronidase (3.2.1.128) or α-D-glucuronidase (EC 3.2.1.139).

Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. As described herein, an expansin-like protein or swollenin-like protein may comprise one or both of such domains and/or may disrupt the structure of cell walls (such as disrupting cellulose structure), optionally without producing detectable amounts of reducing sugars.

A cellulose induced protein, for example the polypeptide product of the cip1 or cip2 gene or similar genes (see Foreman et al., J. Biol. Chem. 278(34), 31988-31997, 2003), a cellulose/cellulosome integrating protein, for example the polypeptide product of the cipA or cipC gene, or a scaffoldin or a scaffoldin-like protein. Scaffoldins and cellulose integrating proteins are multi-functional integrating subunits which may organize cellulolytic subunits into a multi-enzyme complex. This is accomplished by the interaction of two complementary classes of domain, i.e. a cohesion domain on scaffoldin and a dockerin domain on each enzymatic unit. The scaffoldin subunit also bears a cellulose-binding module (CBM) that mediates attachment of the cellulosome to its substrate. A scaffoldin or cellulose integrating protein may comprise one or both of such domains.

A catalase; the term “catalase” means a hydrogen-peroxide: hydrogen-peroxide oxidoreductase (EC 1.11.1.6 or EC 1.11.1.21) that catalyzes the conversion of two hydrogen peroxides to oxygen and two waters. Catalase activity can be determined by monitoring the degradation of hydrogen peroxide at 240 nm based on the following reaction: 2H₂O₂→2H₂O+O₂. The reaction is conducted in 50 mM phosphate pH 7.0 at 25° C. with 10.3 mM substrate (H₂0₂) and approximately 100 units of enzyme per ml. Absorbance is monitored spectrophotometrically within 16-24 seconds, which should correspond to an absorbance reduction from 0.45 to 0.4. One catalase activity unit can be expressed as one micromole of H₂0₂ degraded per minute at pH 7.0 and 25° C.

The term “amylase” as used herein means enzymes that hydrolyze alpha-1,4-glucosidic linkages in starch, both in amylose and amylopectin, such as alpha-amylase (EC 3.2.1.1), beta-amylase (EC 3.2.1.2), glucan 1,4-alpha-glucosidase (EC 3.2.1.3), glucan 1,4-alpha-maltotetraohydrolase (EC 3.2.1.60), glucan 1,4-alpha-maltohexaosidase (EC 3.2.1.98), glucan 1,4-alpha-maltotriohydrolase (EC 3.2.1.116) and glucan 1,4-alpha-maltohydrolase (EC 3.2.1.133), and enzymes that hydrolyze alpha-1,6-glucosidic linkages, being the branch-points in amylopectin, such as pullulanase (EC 3.2.1.41) and limit dextinase (EC 3.2.1.142).

A composition for use in the processes as described herein may be composed of enzymes from (1) commercial suppliers; (2) cloned genes expressing enzymes; (3) broth (such as that resulting from growth of a microbial strain in media, wherein the strains secrete proteins and enzymes into the media; (4) cell lysates of strains grown as in (3); and/or (5) plant material expressing enzymes. Different enzymes in a composition of the invention may be obtained from different sources.

The enzymes can be produced either exogenously in microorganisms, yeasts, fungi, bacteria or plants, then isolated and added, for example, to lignocellulosic material. Alternatively, the enzyme may be produced in a fermentation that uses (pretreated) lignocellulosic material (such as corn stover or wheat straw) to provide nutrition to an organism that produces an enzyme(s). In this manner, plants that produce the enzymes may themselves serve as a lignocellulosic material and be added into lignocellulosic material.

In the uses and processes described herein, the components of the compositions described above may be provided concomitantly (i.e. as a single composition per se) or separately or sequentially.

Lignocellulosic material as used herein includes any lignocellulosic and/or hemicellulosic material. Lignocellulosic material suitable for use in the processes as described herein includes biomass, e.g. virgin biomass and/or non-virgin biomass such as agricultural biomass, commercial organics, construction and demolition debris, municipal solid waste, waste paper and yard waste. Common forms of biomass include trees, shrubs and grasses, wheat, wheat straw, sugar cane, cane straw, sugar cane bagasse, switch grass, miscanthus, energy cane, corn, corn stover, corn husks, corn cobs, corn fiber, corn kernels, canola stems, soybean stems, sweet sorghum, products and by-products from milling of grains such as corn, wheat and barley (including wet milling and dry milling) often called “bran or fibre”, distillers dried grains, as well as municipal solid waste, waste paper and yard waste. The biomass can also be, but is not limited to, herbaceous material, agricultural residues, forestry residues, municipal solid wastes, waste paper, and pulp and paper mill residues. “Agricultural biomass” includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, fruit peels, vines, sugar beet pulp, wheat midlings, oat hulls, and hard and soft woods (not including woods with deleterious materials). In addition, agricultural biomass includes organic waste materials generated from agricultural processes including farming and forestry activities, specifically including forestry wood waste. Agricultural biomass may be any of the afore-mentioned singularly or in any combination or mixture thereof.

The enzyme composition used in the process as described herein can extremely effectively hydrolyze lignocellulosic material, for example corn stover, wheat straw, cane straw, and/or sugar cane bagasse, which can then be further converted into a product, such as ethanol, biogas, butanol, a plastic, an organic acid, a solvent, an animal feed supplement, a pharmaceutical, a vitamin, an amino acid, an enzyme or a chemical feedstock. Additionally, intermediate products from a process following the hydrolysis, for example lactic acid as intermediate in biogas production, can be used as building block for other materials.

In an embodiment the amount of protein (i.e. enzyme composition protein as determined by biuret assay (see e.g. Example 1)) added in step (i) (of the hydrolysis process as described herein) is from 1 to 40 mg/g glucan in the pretreated ignocellulosic material. Preferably, the amount of protein added in step (i) is from 2 to 30 mg/g glucan in the pretreated ignocellulosic material, from 3 to 20 mg/g glucan in the pretreated lignocellulosic material, from 4 to 18 mg/g glucan in the pretreated lignocellulosic material and preferably from 5 to 15 mg/g glucan in the pretreated lignocellulosic material.

In an embodiment the amount of LPMO protein (as determined by TCA-biuret assay (see e.g. Example 1)) added in step (iii) (of the hydrolysis process as described herein) is from 0.01 to 20 mg/g glucan in the pretreated lignocellulosic material. Preferably, the amount of LPMO protein added in step (iii) is from 0.02 to 15 mg/g glucan in the pretreated ignocellulosic material, from 0.05 to 10 mg/g glucan in the pretreated lignocellulosic material, from 0.1 to 8 mg/g glucan in the pretreated ignocellulosic material and preferably from 0.2 to 5 mg/g glucan in the pretreated lignocellulosic material.

The amount of glucan in the pretreated lignocellulosic material is measured according to the method described by Carvalho de Souza et al. (Carbohydrate Polymers, 95 (2013) 657-663).

The pH during the enzymatic hydrolysis may be chosen by the skilled person. In an embodiment the pH during the hydrolysis is from 3.0 to 6.5, from 3.5 to 6.0, preferably from 4.0 to 5.0.

In an embodiment the enzymatic hydrolysis is done at a temperature from 40° C. to 90° C., from 45° C. to 80° C., from 50° C. to 70° C., from 55° C. to 65° C.

In an embodiment the enzymatic hydrolysis is conducted until 70% or more, 80% or more, 85% or more, 90% or more, 92% or more, 95% or more of available sugar in the lignocellulosic material is released.

Significantly, an enzymatic hydrolysis process as described may be carried out using high levels of dry matter of the lignocellulosic material. In an embodiment the dry matter content is 5 wt % or higher, 6 wt % or higher, 7 wt % or higher, 8 wt % or higher, 9 wt % or higher, 10 wt % or higher, 11 wt % or higher, 12 wt % or higher, 13 wt % or higher, 14 wt % or higher, 15 wt % or higher, 16 wt % or higher, 17 wt % or higher, 18 wt % or higher, 19 wt % or higher, 20 wt % or higher, 21 wt % or higher, 22 wt % or higher, 23 wt % or higher, 24 wt % or higher, 25 wt % or higher, 26 wt % or higher, 27 wt % or higher, 28 wt % or higher, 29 wt % or higher, 30 wt % or higher, 31 wt % or higher, 32 wt % or higher, 33 wt % or higher, 34 wt % or higher, 35 wt % or higher, 36 wt % or higher, 37 wt % or higher, 38 wt % or higher or 39 wt % or higher. In an embodiment the dry matter content of the enzymatic hydrolysis is from 5 wt %-40 wt %, from 6 wt %-38 wt %, from 7 wt %-36 wt %, from 8 wt %-34 wt %, from 9 wt %-32 wt %, from 10 wt %-30 wt %, from 11 wt %-28 wt %, from 12 wt %-26 wt %, from 13 wt %-24 wt %, from 14 wt %-22 wt %, from 15 wt %-20 wt % As described above, the present invention also relates to a process for the preparation of a fermentation product from lignocellulosic material, comprising the steps of (a) performing a process for the preparation of a sugar product from lignocellulosic material as described above, (b) fermenting the sugar product to obtain the fermentation product; and (c) optionally, recovering the fermentation product.

In an embodiment the fermentation (i.e. step b) is performed in one or more bioreactors. In an embodiment the fermentation is done by an alcohol producing microorganism to produce alcohol. The fermentation by an alcohol producing microorganism to produce alcohol can be done in the same bioreactor wherein the enzymatic hydrolysis is performed. Alternatively, the fermentation by an alcohol producing microorganism to produce alcohol can be performed in one or more separate bioreactors.

In an embodiment the fermentation is done by a yeast. In an embodiment the alcohol producing microorganism is a yeast. In an embodiment the alcohol producing microorganism is able to ferment at least a C5 sugar and at least a C6 sugar.

In a further aspect, the invention thus includes fermentation processes in which a microorganism is used for the fermentation of a carbon source comprising sugar(s), e.g. glucose, L-arabinose and/or xylose. The carbon source may include any carbohydrate oligo- or polymer comprising L-arabinose, xylose or glucose units, such as e.g. lignocellulose, xylans, cellulose, starch, arabinan and the like. For release of xylose or glucose units from such carbohydrates, appropriate carbohydrases (such as xylanases, glucanases, amylases and the like) may be added to the fermentation medium or may be produced by the modified host cell. In the latter case, the modified host cell may be genetically engineered to produce and excrete such carbohydrases. An additional advantage of using oligo- or polymeric sources of glucose is that it enables to maintain a low(er) concentration of free glucose during the fermentation, e.g. by using rate-limiting amounts of the carbohydrases. This, in turn, will prevent repression of systems required for metabolism and transport of non-glucose sugars such as xylose. In a preferred process the modified host cell ferments both the L-arabinose (optionally xylose) and glucose, preferably simultaneously in which case preferably a modified host cell is used which is insensitive to glucose repression to prevent diauxic growth. In addition to a source of L-arabinose, optionally xylose (and glucose) as carbon source, the fermentation medium will further comprise the appropriate ingredient required for growth of the modified host cell. Compositions of fermentation media for growth of microorganisms such as yeasts or filamentous fungi are well known in the art.

The fermentation process may be an aerobic or an anaerobic fermentation process. An anaerobic fermentation process is herein defined as a fermentation process run in the absence of oxygen or in which substantially no oxygen is consumed, preferably less than 5, 2.5 or 1 mmol/L/h, more preferably 0 mmol/L/h is consumed (i.e. oxygen consumption is not detectable), and wherein organic molecules serve as both electron donor and electron acceptors. In the absence of oxygen, NADH produced in glycolysis and biomass formation, cannot be oxidised by oxidative phosphorylation. To solve this problem many microorganisms use pyruvate or one of its derivatives as an electron and hydrogen acceptor thereby regenerating NAD⁺. Thus, in a preferred anaerobic fermentation process pyruvate is used as an electron (and hydrogen acceptor) and is reduced to fermentation products such as ethanol, lactic acid, 3-hydroxy-propionic acid, acrylic acid, acetic acid, succinic acid, citric acid, malic acid, fumaric acid, an amino acid, 1,3-propane-diol, ethylene, glycerol, butanol, a β-lactam antibiotic and a cephalosporin. In a preferred embodiment, the fermentation process is anaerobic. An anaerobic process is advantageous, since it is cheaper than aerobic processes: less special equipment is needed. Furthermore, anaerobic processes are expected to give a higher product yield than aerobic processes. Under aerobic conditions, usually the biomass yield is higher than under anaerobic conditions. As a consequence, usually under aerobic conditions, the expected product yield is lower than under anaerobic conditions.

In another embodiment, the fermentation process is under oxygen-limited conditions. More preferably, the fermentation process is aerobic and under oxygen-limited conditions. An oxygen-limited fermentation process is a process in which the oxygen consumption is limited by the oxygen transfer from the gas to the liquid. The degree of oxygen limitation is determined by the amount and composition of the ingoing gas flow as well as the actual mixing/mass transfer properties of the fermentation equipment used. Preferably, in a process under oxygen-limited conditions, the rate of oxygen consumption is at least 5.5, more preferably at least 6 and even more preferably at least 7 mmol/L/h. In an embodiment the alcohol fermentation process is anaerobic.

The fermentation process is preferably run at a temperature that is optimal for the microorganism used. Thus, for most yeasts or fungal cells, the fermentation process is performed at a temperature which is less than 42° C., preferably 38° C. or lower. For yeast or filamentous fungal host cells, the fermentation process is preferably performed at a temperature which is lower than 35, 33, 30 or 28° C. and at a temperature which is higher than 20, 22, or 25° C. In an embodiment the alcohol fermentation step is performed between 25° C. and 35° C.

In an embodiment the fermentations are conducted with a fermenting microorganism. In an embodiment of the invention, the alcohol (e.g. ethanol) fermentations of C5 sugars are conducted with a C5 fermenting microorganism. In an embodiment of the invention, the alcohol (e.g. ethanol) fermentations of C6 sugars are conducted with a C5 fermenting microorganism or a commercial C6 fermenting microorganism. Commercially available yeast suitable for ethanol production include, but are not limited to, BIOFERM™ AFT and XR (NABC-North American Bioproducts Corporation, GA, USA), ETHANOL RED™ yeast (Fermentis/Lesaffre, USA), FALI™ (Fleischmann's Yeast, USA), FERMIOL™ (DSM Specialties), GERT STRAND™ (Gert Strand AB, Sweden), and SUPERSTART™ and THERMOSACC™ fresh yeast (Ethanol Technology, WI, USA).

In an embodiment the alcohol producing microorganism is a microorganism that is able to ferment at least one C5 sugar. Preferably, it also is able to ferment at least one C6 sugar. In an embodiment the application describes a process for the preparation of ethanol from lignocellulosic material, comprising the steps of (a) performing a process for the preparation of a sugar product from lignocellulosic material as described above, (b) fermentation of the sugar product to produce ethanol; and (c) optionally, recovery of the ethanol. The fermentation can be done with a microorganism that is able to ferment at least one C5 sugar.

The alcohol producing microorganisms may be a prokaryotic or eukaryotic organism. The microorganism used in the process may be a genetically engineered microorganism. Examples of suitable alcohol producing organisms are yeasts, for instance Saccharomyces, e.g. Saccharomyces cerevisiae, Saccharomyces pastorianus or Saccharomyces uvarum, Hansenula, Issatchenkia, e.g. Issatchenkia orientalis, Pichia, e.g. Pichia stipites or Pichia pastoris, Kluyveromyces, e.g. Kluyveromyces fagilis, Candida, e.g. Candida pseudotropicalis or Candida acidothermophilum, Pachysolen, e.g. Pachysolen tannophilus or bacteria, for instance Lactobacillus, e.g. Lactobacillus lactis, Geobacillus, Zymomonas, e.g. Zymomonas mobilis, Clostridium, e.g. Clostridium phytofermentans, Escherichia, e.g. E. coli, Klebsiella, e.g. Klebsiella oxytoca. In an embodiment the microorganism that is able to ferment at least one C5 sugar is a yeast. In an embodiment, the yeast belongs to the genus Saccharomyces, preferably of the species Saccharomyces cerevisiae. The yeast, e.g. Saccharomyces cerevisiae, used in the processes according to the present invention is capable of converting hexose (C6) sugars and pentose (C5) sugars. The yeast, e.g. Saccharomyces cerevisiae, used in the processes according to the present invention can anaerobically ferment at least one C6 sugar and at least one C5 sugar. For example, the yeast is capable of using L-arabinose and xylose in addition to glucose anaerobically. In an embodiment, the yeast is capable of converting L-arabinose into L-ribulose and/or xylulose 5-phosphate and/or into a desired fermentation product, for example into ethanol. Organisms, for example Saccharomyces cerevisiae strains, able to produce ethanol from L-arabinose may be produced by modifying a host yeast introducing the araA (L-arabinose isomerase), araB (L-ribuloglyoxalate) and araD (L-ribulose-5-P4-epimerase) genes from a suitable source. Such genes may be introduced into a host cell in order that it is capable of using arabinose. Such an approach is given is described in WO2003/095627. araA, araB and araD genes from Lactobacillus plantarum may be used and are disclosed in WO2008/041840. The araA gene from Bacillus subtilis and the araB and araD genes from Escherichia coli may be used and are disclosed in EP1499708. In another embodiment, araA, araB and araD genes may derived from of at least one of the genus Clavibacter, Arthrobacter and/or Gramella, in particular one of Clavibacter michiganensis, Arthrobacter aurescens, and/or Gramella forsetii, as disclosed in WO 2009011591. In an embodiment, the yeast may also comprise one or more copies of xylose isomerase gene and/or one or more copies of xylose reductase and/or xylitol dehydrogenase.

The yeast may comprise one or more genetic modifications to allow the yeast to ferment xylose. Examples of genetic modifications are introduction of one or more xylA-gene, XYL1 gene and XYL2 gene and/or XKS1-gene; deletion of the aldose reductase (GRE3) gene; overexpression of PPP-genes TAL1, TKL1, RPE1 and RKI1 to allow the increase of the flux through the pentose phosphate pathway in the cell. Examples of genetically engineered yeast are described in EP1468093 and/or WO2006/009434.

An example of a suitable commercial yeast is RN1016 that is a xylose and glucose fermenting Saccharomyces cerevisiae strain from DSM, the Netherlands.

In an embodiment, the fermentation process for the production of ethanol is anaerobic. Anaerobic has already been defined earlier herein. In another preferred embodiment, the fermentation process for the production of ethanol is aerobic. In another preferred embodiment, the fermentation process for the production of ethanol is under oxygen-limited conditions, more preferably aerobic and under oxygen-limited conditions. Oxygen-limited conditions have already been defined earlier herein.

Alternatively, to the fermentation processes described above, at least two distinct cells may be used, this means this process is a co-fermentation process. All preferred embodiments of the fermentation processes as described above are also preferred embodiments of this co-fermentation process: identity of the fermentation product, identity of source of L-arabinose and source of xylose, conditions of fermentation (aerobic or anaerobic conditions, oxygen-limited conditions, temperature at which the process is being carried out, productivity of ethanol, yield of ethanol).

Fermentation products that may be produced by the processes of the invention can be any substance derived from fermentation. They include, but are not limited to, alcohol (such as arabinitol, butanol, ethanol, glycerol, methanol, 1,3-propanediol, sorbitol, and xylitol); organic acid (such as acetic acid, acetonic acid, adipic acid, ascorbic acid, acrylic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, maleic acid, malic acid, malonic acid, oxalic acid, oxaloacetic acid, propionic acid, succinic acid, and xylonic acid); ketones (such as acetone); amino acids (such as aspartic acid, glutamic acid, glycine, lysine, serine, tryptophan, and threonine); alkanes (such as pentane, hexane, heptane, octane, nonane, decane, undecane, and dodecane), cycloalkanes (such as cyclopentane, cyclohexane, cycloheptane, and cyclooctane), alkenes (such as pentene, hexene, heptene, and octene); and gases (such as methane, hydrogen (H₂), carbon dioxide (C0₂), and carbon monoxide (CO)). The fermentation product can also be a protein, a vitamin, a pharmaceutical, an animal feed supplement, a specialty chemical, a chemical feedstock, a plastic, a solvent, ethylene, an enzyme, such as a protease, a cellulase, an amylase, a glucanase, a lactase, a lipase, a lyase, an oxidoreductase, a transferase or a xylanase. In a preferred embodiment an alcohol is prepared in the fermentation processes as described herein. In a preferred embodiment ethanol is prepared in the fermentation processes as described herein.

The processes as described herein may comprise recovery of all kinds of products made during the processes including fermentation products such as ethanol. A fermentation product may be separated from the fermentation broth in manner know to the skilled person. Examples of techniques for recovery include, but are not limited to, chromatography, electrophoretic procedures, differential solubility, distillation, or extraction. For each fermentation product, the skilled person will thus be able to select a proper separation technique. For instance, ethanol may be separated from a yeast fermentation broth by distillation, for instance steam distillation/vacuum distillation in conventional way.

In an embodiment the processes as described herein also produce energy, heat, electricity and/or steam.

The beneficial effects of the present invention are found for several ignocellulosic materials and therefore believed to be present for the hydrolysis of all kind of ignocellulosic materials. The beneficial effects of the present invention are found for several enzymes and therefore believed to be present for all kind of hydrolysing enzyme compositions.

### EXAMPLES

### Example 1

### Addition of a Lytic Polysaccharide Monooxygenase (LPMO) Before Start of Aeration

This example shows the effect of adding additional LPMO before aeration on hydrolysis of lignocellulosic material.

Rasamsonia emersonii cellulase cocktail and Rasamsonia emersonii ΔLPMO-cellulase cocktail (i.e. both whole fermentation broths) were produced according to the methods as described in WO2011/000949. Rasamsonia emersonii ΔLPMO strain was made by deleting the gene encoding LPMO (see WO2012/000892) from a Rasamsonia emersonii strain by methods known in the art. Moreover, Rasamsonia emersonii lytic polysaccharide monooxygenase (LPMO) as described in WO2012/000892 and Rasamsonia emersonii beta-glucosidase as described in WO2012/000890 were used in the experiments.

The protein concentration of the LPMO was determined using a TCA-biuret method. In short, bovine serum albumin (BSA) dilutions (0, 1, 2, 5, 8 and 10 mg/ml) were made to generate a calibration curve. Additionally, dilutions of LPMO samples were made with water. Of each diluted sample (of the BSA and the LPMO), 270 μl was transferred into a 10-ml tube containing 830 μl of a 12% (w/v) trichloro acetic acid solution in acetone and mixed thoroughly. Subsequently, the tubes were incubated on ice water for one hour and centrifuged for 30 minutes at 4° C. and 6000 rpm. The supernatant was discarded and pellets were dried by inverting the tubes on a tissue and letting them stand for 30 minutes at room temperature. Next, 3 ml BioQuant Biuret reagent mix was added to the pellet in the tubes and the pellet was solubilized upon mixing followed by addition of 1 ml water. The tubes were mixed thoroughly and incubated at room temperature for 30 minutes. The absorption of the mixtures was measured at 546 nm and a water sample was used as a blank measurement. Dilutions of the LPMO that gave an absorption value at 546 nm within the range of the calibration line were used to calculate the total protein concentration of the LPMO samples via the BSA calibration line.

The protein concentration of the cellulase cocktails was determined using a biuret method. Cocktail samples were diluted on weight basis with water and centrifugated for 5 minutes at >14000×g. Bovine serum albumin (BSA) dilutions (0.5, 1, 2, 5, 10 and 15 mg/ml) were made to generate a calibration curve. Of each diluted protein sample (of the BSA and the cocktail), 200 μl of the supernatant was transferred into a 1.5 ml reaction tube. 800 μl BioQuant Biuret reagent was added and mixed thoroughly. From the same diluted protein sample, 500 μl was added to reaction tube containing a 10 KD filter. 200 μl of the effluent was transferred into a 1.5 ml reaction tube, 800 μl BioQuant Biuret reagent was added and mixed thoroughly. Next, all mixtures (diluted protein samples before and after 10 KD filtration mixed with BioQuant) were incubated at room temperature for at least 30 minutes. The absorption of the mixtures was measured at 546 nm with a water sample used as a blank measurement. Dilutions of the cocktail that gave an absorption value at 546 nm within the range of the calibration line were used to calculate the total protein concentration of the cocktail samples via the BSA calibration line.

Enzymatic beta-glucosidase activity (WBDG) was determined at 37° C. and pH 4.4 using para-nitrophenyl-ß-D-glucopyranoside as substrate. Enzymatic hydrolysis of pNP-beta-D-glucopyranoside resulted in release of para-nitrophenol (pNP) and D-glucose. Quantitatively released para-nitrophenol, determined under alkaline conditions, was a measure for enzymatic activity. After 10 minutes of incubation, the reaction was stopped by adding 1 M sodium carbonate and the absorbance was determined at a wavelength of 405 nm. Beta-glucosidase activity was calculated making use of the molar extinction coefficient of para-nitrophenol. A para-nitro-phenol calibration line was prepared by diluting a 10 mM pNP stock solution in acetate buffer 100 mM pH 4.40 0.1% BSA to pNP concentrations 0.25, 0.40, 0.67 and 1.25 mM. The substrate was a solution of 5.0 mM pNP-BDG in an acetate buffer (100 mM, pH 4.4). To 3 ml substrate, 200 μl of calibration solution and 3 ml 1M sodium carbonate was added. The absorption of the mixture was measured at 405 nm with an acetate buffer (100 mM) used as a blank measurement. The pNP content was calculated using standard calculation protocols known in the art, by plotting the OD₄₀₅ versus the concentration of samples with known concentration, followed by the calculation of the concentration of the unknown samples using the equation generated from the calibration line. Samples were diluted in weight corresponding to an activity between 1.7 and 3.3 units. To 3 ml substrate, preheated to 37° C., 200 μl of diluted sample solution was added. This was recorded as t=0. After 10.0 minutes, the reaction was stopped by adding 3 ml 1M sodium carbonate. The beta-glucosidase activity is expressed in WBDG units per gram enzyme broth. One WBDG unit is defined as the amount of enzyme that liberates one nanomol para-nitrophenol per second from para-nitrophenyl-beta-D-glucopyranoside under the defined assay conditions (4.7 mM pNPBDG, pH=4.4 and T=37° C.).

Acid pretreated corn stover (aCS) was made by incubating corn stover for 6.7 minutes at 186° C. Prior to the heat treatment, the corn stover was impregnated with H₂SO₄ for 10 minutes to set the pH at 2.3 during the pretreatment. The amount of glucan in the pretreated lignocellulosic material was measured according to the method described by Carvalho de Souza et al. (Carbohydrate Polymers, 95 (013) 657-663. The hydrolysis reactions were performed with acid pretreated corn stover (aCS) at a final concentration of 17% (w/w) dry matter. The feedstock solution was prepared via dilution of a concentrated feedstock solution with water. Subsequently, the pH was adjusted to pH 4.5 with a 10% (w/w) NH₄OH solution.

Hydrolysis reactions were done in a stirred, pH-controlled and temperature-controlled closed reactor with a working volume of 1 I. Each hydrolysis was performed and controlled at pH 4.5 and at 62° C. The reaction vessels were filled with the 17% (w/w) feedstock (pH 4.5) and stirred at 150 rpm for 18 hours, while the headspace was continuously refreshed by a flow of nitrogen (100 ml/min) at 62° C. to get the vessel anaerobic. Subsequently, the hydrolysis reactions were started and the following experiments were done:
1) Addition at t=0 hours of (a) a Rasamsonia emersonii cellulase cocktail at a concentration of 7 mg protein/g glucan in the pretreated lignocellulosic material and (b) 836 WBDG units/g glucan in the pretreated lignocellulosic material (control reaction).
2) Addition at t=0 hours of a Rasamsonia emersonii cellulase cocktail at a concentration of 7 mg protein/g glucan in the pretreated lignocellulosic material, 836 WBDG/g glucan in the pretreated lignocellulosic material and 0.7 mg Rasamsonia emersonii LPMO protein/g glucan in the pretreated lignocellulosic material (LPMO protein addition at t=0 hours).
3) Addition at t=0 hours of a Rasamsonia emersonii cellulase cocktail at a concentration of 7 mg protein/g glucan in the pretreated lignocellulosic material, 836 WBDG/g glucan in the pretreated lignocellulosic material and a Rasamsonia emersonii ΔLPMO-cellulase cocktail at a concentration of 0.7 mg protein/g glucan in the pretreated lignocellulosic material (ΔLPMO-cellulase cocktail addition at t=0 hours).

After addition of the enzymes at t=0 hour, each hydrolysis vessel was kept anaerobic for 6 hours, after which the nitrogen flow (100 ml/min) was exchanged by an air flow (100 ml/min) resulting in a dissolved oxygen (DO) level of 5% (0.008 mol/m³) in the reaction mixture as measured by a DO-electrode. The total hydrolysis time was 144 hours.

At the end of the hydrolysis, samples were taken for analysis which were immediately centrifuged for 8 min at 4000×g. The supernatant was filtered over 0.2 μm nylon filters (whatman) and stored at 4° C. until analysis for sugar content as described below.

The sugar concentrations of the diluted samples were measured using an HPLC equipped with an Aminex HPX-87H column according to the NREL technical report NREL/TP-510-42623, January 2008. The results are presented in Table 1.

The data show that it is beneficial to add additional LPMO protein in a hydrolysis process, resulting in 6% increased glucose release as compared to when nothing is additionally spiked or when an equal amount of cellulase cocktail not containing LPMO is spiked.

### Example 2

Addition of a Lytic Polysaccharide Monooxygenase (LPMO) after Start of Aeration

This example shows the effect of adding additional LPMO after start of aeration on hydrolysis of lignocellulosic material.

The experiment was done as described in Example 1 with the proviso that the following experiments were done:
1) Addition at t=0 hours of (a) a Rasamsonia emersonii cellulase cocktail at a concentration of 7 mg protein/g glucan in the pretreated lignocellulosic material and (b) 836 WBDG units/g glucan in the pretreated lignocellulosic material (control reaction).
2) Addition at t=0 hours of a Rasamsonia emersonii cellulase cocktail at a concentration of 7 mg protein/g glucan in the pretreated lignocellulosic material and 836 WBDG/g glucan in the pretreated lignocellulosic material and addition at t=24 hours of 0.7 mg Rasamsonia emersonii LPMO protein/g glucan in the pretreated lignocellulosic material (LPMO protein addition at t=24 hours).
3) Addition at t=0 hours of a Rasamsonia emersonii cellulase cocktail at a concentration of 7 mg protein/g glucan in the pretreated lignocellulosic material and 836 WBDG/g glucan in the pretreated lignocellulosic material and addition at t=24 hours of a Rasamsonia emersonii ΔLPMO-cellulase cocktail at a concentration of 0.7 mg protein/g glucan in the pretreated lignocellulosic material (ΔLPMO-cellulase cocktail addition at t=24 hours).

The results are presented in Table 2. The data clearly show that it is beneficial to add LPMO protein after start of the aeration (12% increased glucose release) as compared to when nothing is additionally spiked or when an equal amount of cellulase cocktail not containing LPMO is spiked after start of aeration. Addition of LPMO protein after start of aeration (12% additional glucose release) is advantageous over addition of LPMO protein before start of aeration (6% additional glucose release).

TABLE 1
Effect of addition of LPMO protein or ΔLPMO-cellulase
cocktail before start of aeration on glucose release as
measured at the end of the hydrolysis process (t = 144 hour).

| Experiment | Glucose release (g/l) |
|---|---|
| No LPMO spiking (control reaction) | 50.9 |
| Spiking of LPMO protein at t = 0 hours | 53.8 |
| Spiking ΔLPMO-cellulase cocktail at t = 0 | 50.9 |

TABLE 2
Effect of addition of LPMO protein or ΔLPMO-cellulase
cocktail after start of aeration on glucose release as
measured at the end of the hydrolysis process (t = 144 hour)

| Experiment | Glucose release (g/l) |
|---|---|
| No LPMO spiking (control reaction) | 54.1 |
| Spiking of LPMO protein at t = 24 hours | 60.6 |
| Spiking ΔLPMO-cellulase cocktail at t = 24 hours | 54.2 |

1. A process for preparation of a sugar product from lignocellulosic material, said process comprising:
a) pretreating the lignocellulosic material,
b) enzymatically hydrolysing the pretreated lignocellulosic material in an enzymatic hydrolysis to obtain the sugar product in a process comprising:
i) first treating the lignocellulosic material with an enzyme composition comprising a lytic polysaccharide monooxygenase and a polypeptide selected from the group consisting of a cellobiohydrolase, an endoglucanase, a beta-glucosidase, a beta-xylosidase, an endoxylanase and any combination thereof, then
ii) adding oxygen to the mixture comprising the lignocellulosic material and the enzyme composition, and thereafter
iii) adding additional lytic polysaccharide monooxygenase to the mixture comprising the lignocellulosic material and the enzyme composition, and
c) optionally, recovering the sugar product.

2. A process for preparation of a fermentation product from lignocellulosic material, comprising:
a) performing a process according to claim 1,
b) fermenting the sugar product to produce the fermentation product; and
c) optionally, recovering the fermentation product.

3. The process according to claim 1, wherein dry matter content of the lignocellulosic material in the enzymatic hydrolysis is from 10-40 wt %.

4. The process according to claim 1, wherein the enzyme composition comprises a lytic polysaccharide monooxygenase and/or the additional lytic polysaccharide monooxygenase is from a fungus.

5. The process according to claim 1, wherein the enzyme composition comprising a lytic polysaccharide monooxygenase and/or the additional lytic polysaccharide monooxygenase is added in the form of a whole fermentation broth of a fungus.

6. The process according to claim 4, wherein the fungus is Rasamsonia.

7. The process according to claim 2, wherein the fermentation is done by a yeast.

8. The process according to claim 1, wherein the enzymatic hydrolysis is done in a bioreactor having a volume of at least 10 m³.

9. The process according to claim 1, wherein the start of (ii) is from 1 to 100 hours after the start of (i).

10. The process according to claim 1, wherein the amount of protein added in (i) is from 1 to 40 mg/g glucan in pretreated lignocellulosic material.

11. The process according to claim 1, wherein the amount of protein added in (iii) is from 0.01 to 20 mg/g glucan in pretreated lignocellulosic material.

12. The process according to claim 1, wherein the ratio of lytic polysaccharide monooxygenase added in (i) to lytic polysaccharide monooxygenase added in (iii) is from 10:1 to 1:10.

13. The process according to claim 1, wherein the pH of the enzymatic hydrolysis is from 3.5 to 5.5.

14. The process according to claim 1, wherein the temperature of the enzymatic hydrolysis is from 50° C. to 70° C.
